(19) United States
(12) Patent Application Publication
(10) Pub. No.: US 2008/0267862 A1
(43) Pub. Date: Oct. 30, 2008

(54) METHOD OF TREATING CANCER

(76) Inventor: DEBATOSH DATTA, Calcutta (IN)

Correspondence Address: NOTARO AND MICHALOS, 100 DUTCH HILL ROAD, SUITE 110, ORANGEBURG, NY, 10962-2100, US

(21) Appl. No.: 12/111,253

(22) Filed: Apr. 29, 2008

Related U.S. Application Data
(60) Provisional application No. 60/914,794, filed on Apr. 30, 2007.

(51) Int. Cl. A61K 31/195; A61K 50/00; A61K 39/395; A61P 35/00

(52) U.S. Cl. 424/1.11; 514/564; 424/130.1; 424/141.1

(57) ABSTRACT

Present invention provides a method for treating cancer which comprises co-administration of an angiogenic agent to enhance the vascular supply within tumors, particularly tumors capable of maintaining their viability under hypoxic conditions, and a anticancer therapy (e.g. chemotherapy and radiation therapy) to which the cancer is susceptible. Induction of angiogenesis increases the delivery of anticancer agents to the hypoxic tumor cells within the tumors, and as a result, improves the effectiveness of the anticancer therapy in eliminating or reducing cancers. The present invention also includes formulations comprising of lysine(L- and/or D-isomers as well as, or, their “activated” version either in isolation or in various combinations, as described, along with additive(s) and one or more chemotherapeutic agent(s) and/or radio-sensitizing agent(s).

### FIELD AND BACKGROUND OF THE INVENTION

The invention relates to a new and improved method for treating cancer. The method of the invention can be used in anticancer therapy to provide controlled induction of angiogenesis and/or vasculogenesis within cancerous tumors that are hypoxic to facilitate delivery of anticancer agent and/or tumor sensitizing agent to the hypoxic tumor cells. Angiogenesis and/or vasculogenesis can be achieved by the method according to the invention by administration of an angiogenic effective amount of an angiogenic agent to sensitize the hypoxic tumor cells to chemotherapy and/or radiation therapy.

Angiogenesis is a process involved in the growth of blood vessels. Angiogenesis is the process by which new vessels are formed from extant capillaries and the factors that regulate this process are important in embryonic development and contribute to pathologic conditions such as tumor growth, diabetic retinopathy, rheumatoid arthritis etc. See U.S. Pat. No. 5,318,957; Yancopoulos et al. (1998) Cell 93:661-4; Folkman et al.(1996) Cell 87:1153-5.

Angiogenesis involves the proliferation of endothelial cells. Endothelial cells line the walls of the vessels, while capillaries are comprised almost entirely of endothelial cells. The angiogenic process involves not only increased endothelial cell proliferation but also comprises a cascade of additional events including protease secretion by endothelial cells degradation of the basement membrane, migration through surrounding matrix, proliferation, alignment, differentiation into tube-like structures, and synthesis of a new basement membrane. See Folkman et al. (1996) Cell 87:1153-5.

Several angiogenic agents with different properties and mechanisms of action are well known in the art, e.g., acidic and basic fibroblast growth factor (FGF), transforming growth factor alpha (TGF-alpha) and beta (TGF-beta), Tumor Necrosis Factor (TNF), Platelet derived growth factor (PDGF), vascular endothelial cell growth factor (VEGF), and angiogenin are potent and well characterized angiogenesis promoting agents. However, the therapeutic applicability of some of these compounds, especially as systemic agents is limited by their potent pleiotropic effects on various cell types.

Angiogenesis has been the focus of intense interest since this process can be exploited to therapeutic advantage. Stimulation of angiogenesis can aid in the healing of wounds, the vascularization of skin grafts, and the enhancement of collateral circulation, where there has been vascular occlusion or stenosis (e.g., to develop a bio-bypass around an obstruction due to coronary, carotid or peripheral arterial occlusion disease). There is an intense interest in the factors that are well tolerated by the subject, and at the same time effective in stimulation of angiogenesis in vascular-compromised tissues.

U.S. Pat. No. 6,417,205 issued to Cooke, et al., described the angiogenic and vasculogenic property of nicotine and other nicotine receptor agonists.

Villablanca studied the effects of nicotine on endothelial DNA synthesis, DNA repair, proliferation and cytotoxicity using cultures of bovine pulmonary artery endothelial cells in vitro. Villablanca (1998) Nicotine stimulates DNA synthesis and proliferation in vascular endothelial cells in-vitro; J. Appl. Physiol. 84:2089-98.

Carty, et al. demonstrated that nicotine stimulates vascular smooth muscle cells to produce fibroblast growth factor, and also upregulated the expression of several matrix metalloproteinases. The investigators proposed that their data demonstrated mechanisms by which smoking may cause atherosclerosis and aneurysms. Carty et al. (1996) Nicotine and cotinine stimulate secretion of basic fibroblast growth factor and affect expression of matrix metalloproteinases in cultured human smooth muscle cells; J. Vasc. Surg. 24:927-35.

Lipid molecules, e.g., spingosine-1-phosphate (spp) has been implicated in angiogenesis and blood vessel maturation. English D., et al. (2002) Biochim Biophys Acta, 1582(1-3):228-39. SPP has been reported to be capable of inducing almost every aspect of angiogenesis.

Katada, et al. described the role of chymase, a serine protease, capable of angiotensin conversion (I to II), in the process of angiogenesis through VEGF upregulation mediated by Ang II. Katada, et al. (2002), J Pharmacol Exp Ther 302(3):949-56.

Sivestre, et al. described the uses of recombinant angiogenic growth factors, e.g., VEGF, FGF, etc. as well as drugs with proangiogenic activity in induction of functional revascularization through angiogenesis. Sivestre, et al. (2002) Arch Mal Coeur Vaiss, 95(3):189-96.

Kamihata, et al. described the role of bone marrow mononuclear cells' implantation in induction of angiogenesis in ischaemic rat heart model. Kamihata, et al. (2001) Circulation 104 (9):1046-52.

Yamamoto, et al. described the potential role of ultrasound therapy in inducing transmyocardial channels resulting in improved perfusion. Yamamoto, et al. (2001) Jpn Circ J, 65(6):565-71.

Kawasuji, et al. described induction of therapeutic angiogenesis with intramyocardial administration of basic fibroblast growth factor. Kawasuji, et al. (2000) Ann Thorac Surg., 69(4) 1155-61.

The roles of various angiogenic growth factors, all protein in nature, has been described by various workers, e.g., Harrigan, et al. (2002) Neurosurgery 50(3):589-98; Edelberg, et al. (2002) Circulation 105(5):608-13; Freedman, et al. (2002) Ann Intern Med 136(1):54-71.

For further discussions of angiogenesis, see, for example:

English, et al. (2002) Lipid mediators of angiogenesis and the signaling pathways they initiate. Biochim Biophys Acta 1582(1-3):228-39.

Katada, et al. (2002) Significance of vascular endothelial cell growth factor upregulation mediated via a Chymasw-Angiotensin dependent pathway during angiogenesis in hamster Sponge Granulomas. J Pharmacol Exp Ther 302(3):949-56.

Sivestre, et al. (2002) Angiogenesis therapy in ischaemic disease. Arch Mal Coeur Vaiss 95(3):189-96.

Kamihata, et al. (2001) Implantation of bone marrow mononuclear cells into ischaemic myocardium enhances collateral perfusion and regional function via side supply of angioblasts, angiogenic ligands and cytokines. Circulation 104(9):1046-52.

Yamamoto, et al. (2001) Potential use of ultrasound in creating transmyocardial channels. Jpn Circ J 65(6):565-71.

Kawasuji, et al. (2000) Therapeutic angiogenesis with intramyocardial administration of basic fibroblast growth factor. Ann Thorac Surg 69(4):1155-61.

Harrigan, et al. (2002) Intraventricular infusion of vascular endothelial growth factor promotes cerebral angiogenesis with minimal brain edema. Neurosurgery 50(3):589-98.

Cuevas, et al. (2000) Electromagnetic therapeutic angiogenesis the next step. Neurol Res 22(4):349-50.

Edelberg, et al. (2002) Platelet-derived growth factor-AB limits the extent of myocardial infraction in a rat model:feasibility of restoring impaired angiogenic capacity in the aging heart. Circulation 105(5):608-13.

Freedmand, et al. (2002) Therapeutic angiogenesis for coronary artery disease. Ann Intern Med 136(1):54-71.

Symes, et al. (2000) Focal angiogenic therapy for myocardial ischaemia. J Card Surg 15(4):283-90.

Chou, et al. (2002) Decreased cardiac expression of vascular endothelial growth factor and its receptors in insulin-resistant and diabetic states: a possible explanation for impaired collateral formation in cardiac tissue. Circulation 105(3):373-9.

Hartlapp, et al. (2001) Fibrocytes induce an angiogenic phenotype in cultured endothelial cells and promote angiogenesis in-vivo. FASEB J 15(12):2215-24.

Epstein, et al. (2001) Therapeutic interventions for enhancing collateral development by administration of growth factors: basic principles, early results and potential hazards. Cardiovasc Res 49(3):532-42.

Certain types of tumors are characteristically hypoxic in nature and minimally invasive or non-metastatic. Examples of such tumors include uterine cervical carcinoma and squamous cell carcinoma of the head and neck. The bulk of the matrix of these tumors is hypoxic. This hypoxic results from reduced blood supply to the bulk or core of the tumors. Consequently, the limited bioavailability of the anticancer drugs in the hypoxic core of the tumors causes the hypoxic tumor cells to be or become refractory to radiotherapy and/or the therapeutic action(s) of chemotherapeutic drugs.

Many strategies and compounds have been developed to enhance the cancer-fighting effects of cancer therapy drugs or treatments for cancers that contain hypoxic tumor cells. U.S. Pat. No. 4,889,525, issued to Yuhas, et al., describes sensitizing hypoxic tumor cells to radiation therapy and/or chemotherapy by administering an oxygen carry perfluoro compound. U.S. Pat. No. 6,979,675, issued to Tidmarsh, describes that hypoxic tumor cells are susceptible to a treatment which combines 2-DG and one or more anticancer agents. U.S. Pat. No. 6,121,263, issued to Brown, describes a combination therapy which comprises administration of a benzotriazine chemotherapy agent that is selectively cytotoxic to hypoxic cancer cells and a chemotherapy agent that target normally oxygenated cancer cells.

There is still a need for a method for enhancing the effectiveness of cancer therapies in eliminating hypoxically radioresistant and/or chemotherapy-resistant cancer cells.

### SUMMARY OF THE INVENTION

According to the present invention, there is provided a method for treating cancer of facilitating cancer therapy through administration of one or more angiogenic agents to selectively induce angiogenesis in a tumor or a portion thereof which, in particular, is growing or capable of maintaining its viability under hypoxic conditions. Some illustrative, non-limiting examples of suitable angiogenic agents include lysine, lysine derivative, lysine oligomer (preferably up to mol. wt. 1000) and lysine analogue (e.g., d-lysine). The method of the invention facilities the delivery of therapeutic agents to the cancer site which augments the tumorcidal effects of anticancer therapy or one or more of the therapeutic agents employed in the treatment (i.e., to which the cancer is susceptible).

According to an embodiment of this invention there is provided a formulation for treating carcinoma comprising lysine, its enantiomer, oligomer, analogue or derivative thereof in admixture with chemotherapeutic and/or radiotherapy sensitizing agents.

In further embodiment of this invention, a pharmaceutical formulation with lysine or an enantiomer, oligomer, analogue or derivative thereof can be co-administered with chemotherapy and/or radiotherapy employed in the treatment or mitigation of carcinoma.

Also in accordance with the present invention, there is provided a method of controlling or enhancing angiogenesis in the hypoxic region of a tumor with no, or very limited, adverse effects.

Also in accordance with the present invention, there is provided a method of treating a tumor or a portion thereof growing or capable of maintaining its viability under hypoxic conditions comprising and administering an angiogenic agent to a subject, such as a human being, having the tumor in an amount effective to controllably induce vascularization or enhance the vascular network within the tumor and administering a tumor sensitizing agent to the subject in an amount effective to produce an increase in the sensitivity of the hypoxic tumor cells in the tumor to the tumorcidal effect of the cancer therapy tailored to treat the tumor. Induction of angiogenesis within the tumor increases the bioavailability of the therapeutic agents, such as tumor sensitizing and/or tumor killing agents, to the hypoxic tumor cells present in the tumor and improves the effectiveness of the cancer therapy (i.e., to which the tumor is susceptible) in reducing and/or eliminating the tumor from the subject. In a preferred embodiment, the amount of angiogenic agent administered to promote the desired degree to angiogenesis within the hypoxic region of the tumor reduces the amount of therapeutic agents otherwise required to achieve a therapeutic cytotoxic effect on the hypoxic tumor cells.

Also in accordance with the present invention, there is provided a method of treating a tumor in a subject comprising administering an angiogenic agent to the subject in an amount effective to increase or promote a desired degree of vascularization in a region of the tumor growing or capable of maintaining its viability under hypoxic conditions and administering a therapeutically effective amount of one or more anticancer therapeutic agents to which the tumor is susceptible to the subject. The therapeutic agent can be any suitable chemotherapeutic agent, monoclonal antibody, polyclonal antibody, or two or more of the foregoing. Preferably, the amount of angiogenic agent administered to the subject reduces the amount of therapeutic agents otherwise required to achieve a therapeutic cytotoxic effect on the hypoxic tumor cells.

The various features of novelty which characterize the invention are pointed out with particularly in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIGS. 1A to 1D shows the different structures of the essential amino acid lysine. The structure of the “native” or “ordinary” form of the essential amino acid lysine is shown in FIGS. 1A and 1B. The L- and D-enantiomers of lysine are different by having mirror image orientation of the same groups and atoms around the chiral carbon atom. The “activated” form is the protonated version of the molecule, e.g., in conditions of lack of oxygen/blood supply to any tissue (FIGS. 1C and 1D). Protonation can take place at the two terminal—NH₂ groups, irrespective of the —CH₂-chain length. Note that the two amino groups at the two ends of the molecule which can become protonated, e.g., in situations of ischaemia (e.g., where tissue hydrogen ion concentration goes high to various levels depending on the degree of ischaemia) and can act as the binding sites for the growth factor(s)/angiogenic factor(s) on one end and to the receptor(s) on the other end. There is a concentration window for the molecule(s) to act as the molecular bridge.

FIG. 2 shows oligo-lysine (mol.wt. of about 1000) structure modeling in a non-aqueous minimum energy environment showing the amino groups projected at either ends “in a pack” (the possible binding sites, as explained above). Enhanced activity of the lysine/activated lysine molecule can be attributed to the conformational distribution of amino groups/protonated amino groups in space as may be evidenced from the above figure.

FIG. 3 shows cell culture photographs of d-Lysine (about 10 mcg/ml of added load, in addition to the usual load of l-lysine, whatever was there in the media as the metabolic requirement) mediated cellular expansion in culture. The d-enantiomer of the essential amino acid was equally effective (as compared to the I-variety), in expanding cells in-vitro as well as in-vivo.

FIG. 4 shows histopathology exhibiting extensive angiogenic response in Lysine Monohydrochloride (LMH) treated glandular structure, wherein angiogenic response is shown with arrows.

FIGS. 5-9 show histopathology of Lysine Monohydrochloride (LMH) treated tumor tissue showing huge angiogenic and massive blood channel formation shown with arrow heads.

FIGS. 10-11 show the control in request of squamous cell carcinoma section, depicting total absence of vascular supply.

### DETAILED DESCRIPTION OF PREFERRED EMBODIMENTS

Before the present invention is described, it is to be understood that this invention is not limited to particular methodologies (e.g., modes of administration) or specific compositions described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art of which this invention belongs. Although any methods and materials similar or equivalent to those described herein can be used in the practice or testing of the present invention, the preferred methods and materials are now described. All publications mentioned in this application are incorporated by the reference in their entireties to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

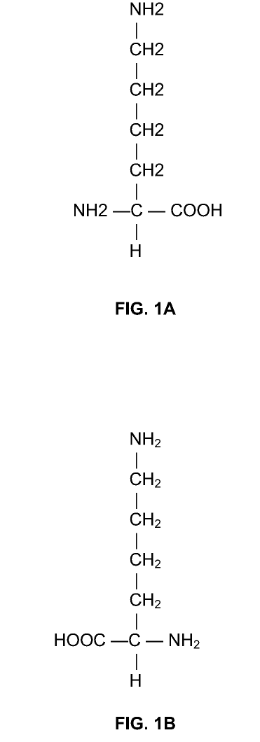

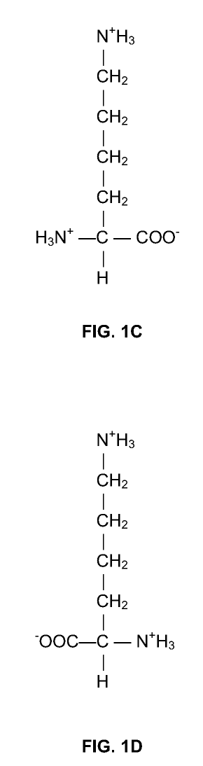

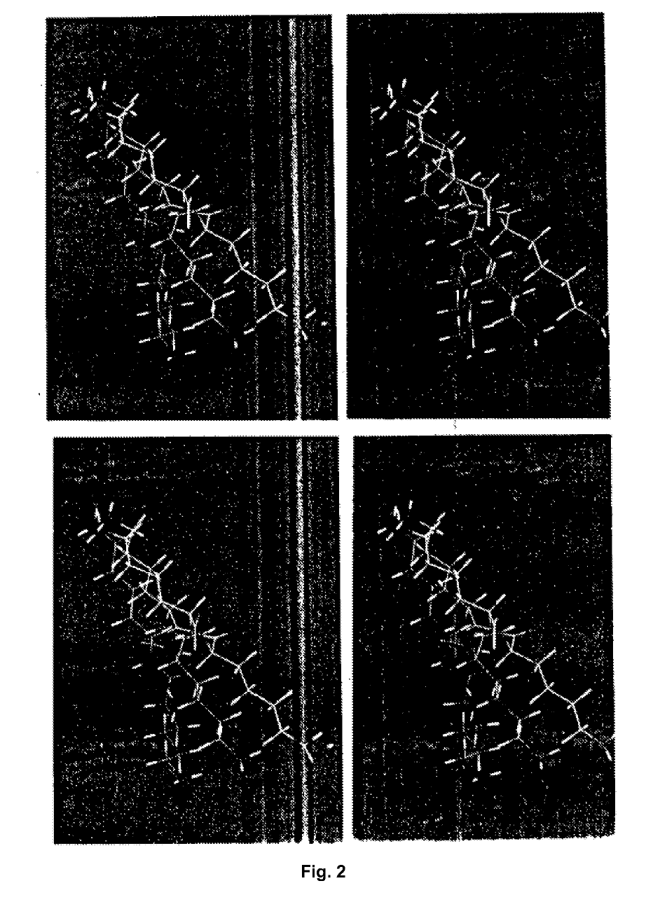

Non-limiting examples of suitable angiogenic agents or angiogenic inducing agents include the naturally occurring cationic essential amino acid known as lysine and its d-isomer, either in it's/their native form(s) or in “activated” form(s) where the terminal amino group(s) at each end of the molecule(s) is/are protonated. Lysine is an off-white/white, dry powder/partly granular/amorphous solid, having a mol. wt. of about 150. Lysine, including its various protonated and isomeric forms, may be isolated and purified from nature or synthetically produced in any manner. Different lysine structures are shown in FIGS. 1A to 1D.

Other suitable angiogenic agents or angiogenic inducing agents include the commonly occurring salts of lysine or the various forms of lysine referred to above, such as hydrochloride, hydrobromide, hydroiodide, nitrate, sulfate or bisulfate phosphate or acid phosphate, acetate, lactate, citrate or acid citrate tartrate, bitartrate, succinate, maleate, fumerate, gluconate, saccharate, benzoate, methanesulfonate, ethanesulfonate, benzenesulfonate, p-toluene sulfonate, camphorate and pamoate salts.

Further illustrative examples of suitable angiogenic agents or angiogenic promoting agents include any pharmacologically acceptable derivative or metabolite of lysine or structurally similar molecule(s) which exhibit(s) pharmacotherapeutic properties similar to lysine, including its/their oligomers (e.g., mol. wt. of about 500-2500). Such derivatives, metabolites and derivatives of metabolites and structurally homologous molecules may be known in the art, and can include, but are not necessarily limited to ornithinine, putrescine, cadaverine, arginine or pharamaceutically acceptable salts thereof.

Examples of lysine derivatives includes but is not limited to d-lysine and lysine oligomers, preferably having mol. wt. of up to or around about 500 to 2500.

Further examples of lysine include its “activated” form, which is accentuated or activated in-situ after application or administration predominantly under anaerobic conditions to bring about an angiogenic response.

The terms “treatment”, “treating” and the like are used herein to generally mean obtaining a desired pharmacologic and/or physiologic effect, e.g., stabilization, remission, regression, shrinkage or decreased volume of cancerous tissue or cell. The effect may be prophylactic in terms of completely or partially preventing a disease or symptom thereof and/or may be therapeutic in terms of a partial or complete cure for a disease and/or adverse effect attributable to the disease.

“Treatment” as used herein covers any treatment of a disease in a mammal, particularly a human, and includes but is not limited to: a) preventing a disease (e.g., cancer) or condition from occurring in a subject who may be predisposed to the disease but has not yet been diagnosed as having it; b) inhibiting the disease, e.g., arresting its development; or c) relieving the disease, i.e., causing regression of the disease (e.g., reduction in tumor volume).

“Hypoxic conditions” as used herein include but are not limited to the physiological conditions of cancer where the inappropriate cell proliferation deprives surrounding tissue of oxygen.

In the context of the present invention, stimulation of angiogenesis is employed for subject having a disease or condition amenable to treatment by increasing vascularity and increasing blood flow. The present invention is particularly directed to a combination therapy for the treatment of cancer, particularly cancer growing or capable of maintaining its viability under hypoxic conditions, with an angiogenic agent and at least one of an anticancer therapeutic agent and a therapeutic radiation having anticancer effect.

By the term effective amount, it is understood that with respect to for example angiogenic agent or angiogenic inducing agent, an amount effective to facilitate a desired therapeutic effect, e.g., a desired level of angiogenic and/or vasculogenic stimulation, is contemplated. The precise desired level of angiogenic and/or vasculogenic stimulation will vary according to the condition to be treated. With respect to for example chemotherapeutic agent, a chemotherapeutically effective amount is contemplated. With respect to for example therapeutic radiation, a therapeutically effective amount of radiation is contemplated.

The present invention is based on an improvement over the inventor's earlier discovery that lysine, without the presence of any other angiogenic factor(s)/angiogenic growth factor(s)/vasculogenic/granulation tissue inducing agent(s), is capable of inducing angiogenesis in ischaemic tissues. The inventor's initial inquiries were directed towards finding new chemical additives in a high-density-culture system, in-vitro, where the objective was to expand viable hybridoma cell mass to the maximum possible at the least possible time (lag phase minimization) and maintaining the viable cell mass in culture for the maximum possible time period, in both fed-batch as well as batch cultures. The final goal was to get highest possible yields of monoclonal antibody (Mab) in the said high-density culture. Monomeric lysine was observed to expand the biomass (cell population) at the least possible time compared to other physico-chemical means (Datta, D et al. 1997). Because of its ability to expand cells in-vitro, the cationic amino acid was taken, along with its d-isomer and short oligomer (m.w. up to or around 500-2500) to in-vivo experimentation and clinical conditions, for controlled regeneration of cells (in clinical conditions), where in-situ regeneration of cells was centrally important along with angio-neogenesis/vasculogensis. Because the molecule has been proposed to have an indirect cell surface bridging role(s), between the cell surface receptors and the growth factor(s) (including the angiogenic factors), derived from the circulating serum protein pool, cellular expansion, migration (of the endothelial cells, forming the very early angiogenic buds) was found to be extremely rapid, reproducible and controlled (Datta, D, Unpublished results).

The inventor has now discovered that induction of angiogenesis within the hypoxic region of tumors provides the basis of a new therapeutic approach to enhance the therapeutic action of drugs and radiation therapies used in the treatment of cancer.

Employing lysine or its isomer or its oligomer as an angiogenic agent has significant advantages over the current candidates as the basis of therapeutic angiogenesis. Moreover, the pharmacology and pharmacokinetics of the essential amino acid has already been well documented and methods for systemic as well as local delivery have been investigated. Processes for the manufacture of lysine and lysine oligomers are also well characterized. Furthermore, these small molecules, particularly the l-variety (l-lysine) is synthesized easily, has no shelf-life problem, extremely stable, highly biocompatible with no known toxicity/side effects even in very high loading doses and is least costly compared to all the other currently known angiogenic agents/factors (which are all mostly proteins or peptides in their native form).

Accordingly, the invention encompasses methods and compositions for stimulation of angiogenesis and/or vasculogenesis by administration of the essential amino acid, its isomer and oligomer. Of particular interest is the stimulation of angiogenesis and/or vasculogenesis in-vivo, to effect increase in blood flow, increased capillary density, and/or increased vascularity within, adjacent, or around an hypoxic site, e.g., regions of tumors that are hypoxic or have reduced or lack uniform vascular supply.

The preferred methods of the invention stimulate angiogenesis to sensitize tumors to the therapeutic actions of cancer therapy. This can be accomplished by administration of a cationic amino acid, particularly lysine, lysine isomer and/or oligomer. Methods for production of the essential amino acid and derivatives, as mentioned, are well known in the art. The preferred method of treating tumors of the invention is accomplished by administration of a chemotherapy and/or a radiation therapy effective to eliminate or reduce the sensitized tumors or being the tumors into remission.

Additional bi-amino compounds, similar structurally to the cationic amino acid of the present invention, include but are not necessarily limited to ornithine, putrescine, cadaverine, arginine, which can be provided in a herbal preparation, either in isolated form (e.g., separated or partially separated from the materials that naturally accompany the said compounds).

Further bi-amino compounds of interest, analogous to the cationic amino acid of the present invention, can be readily identified by the ability of the candidate molecule(s) or their combinations, to stimulate angiogenesis/vasculogenesis in-vivo.

### Pharmaceutical Compositions

Upon reading the present specification, the ordinary skilled artisan will appreciate that the pharmaceutical compositions comprising lysine and derivatives described herein can be provided in a wide variety of formulations. More particularly, the cationic amino acid can be formulated into pharmaceutical compositions by combination with appropriate pharmaceutically acceptable carriers or diluents and may be formulated into preparations in solid, semi-solid (e.g., gel), liquid or gaseous forms, such as tablet, capsules, powders, granules, ointments, solutions, suppositories, injections, inhalants and aerosols. The amino acid, being a naturally-occurring compound, can be formulated as a pharmaceutical composition or an herbal preparation.

The lysine formulation (with or without its isomer and/or oligomer) used will vary according to the condition or disease to be treated, the route of administration, the amount of the active ingredients(s) to be administered, and other variables that will be readily appreciated by the ordinary skilled artisan. In general and as discussed in more detail below, administration of the essential amino acid/derivative(s) or formulation can be either systemic or local, and can be achieved in various ways, including but not necessarily limited to, administration by a route that is parenteral, intravenous, intra-arterial, intrapericardial, intramuscular, intraperitoneal, transdermal, transcutaneous, subdermal, intradermal, oral and intrapulmonary, etc.

In pharmaceutical dosage forms, the cationic amino acid, its derivatives or isomers, or any combination of the foregoing may be administered in the form of its/their pharmaceutically acceptable salts or it/they may also be used alone or in appropriate association, as well as in combination, with other pharmaceutically active compounds.

Therapeutic formulation using cationic amino acid, its derivatives or isomers or any combination referred to herein may have the contents of the active ingredient varying in a manner between 4 and 15 g per day, along with at least on sensitizing agent as described herein, the content of which may vary between 150 mg and 1000 mg, depending on the stage of progress and/or physical condition of the person being treated for cancer.

The following methods and excipients are merely exemplary and are in no way limiting.

The amino acid/derivative(s) can be formulated into preparations for injection by dissolving, suspending or emulsifying them in an aqueous or non-aqueous solvent, such as vegetable or other similar oils, synthetic aliphatic acid glycerides, esters of higher aliphatic acids or propylene glycol, and if desired with conventional additives such as solubilizers, isotonic agents, suspending agents, emulsifying agents, stabilizers and preservatives.

Formulations suitable for topical, transcutaneous, and transdermal administration, e.g., to administer the amino acid/derivative directly to a tumor site, may be similarly prepared through use of appropriate suspending agents, solubilizers, thickening agents stabilizers and preservatives. Topical formulations may also be utilized with a means to provide continuous administration of the amino acid/derivative(s) by, for example, incorporation into slow release pellets or controlled-release patches.

The amino acid/derivative can also be formulated in a biocompatible gel, which gel can be applied topically or implanted (e.g., to provide for sustained release of the amino acid/derivative at an internal treatment site.)

For oral preparations the amino acid/derivative(s) can be used alone or in combination with appropriate additives to make tablets, powders, granules, capsules for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The amino acid/derivatives can be utilized in aerosol formulation to be administered via inhalation. The compounds of the preset invention can be formulated into pressurized acceptable propellants such as dichlorodifluoromethane, propane, nitrogen and the like.

Furthermore, the amino acid and/or derivative(s) can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs and suspensions may be provided wherein each dosage unit, for example, teaspoonful, tablespoonful, tablet or suppository, contains a predetermined amount of the composition containing one or more active ingredients. Similarly, unit dosage forms for injection or intravenous administration may comprise the amino acid derivative(s) in a composition as a solution in sterile water, normal saline or another pharmaceutically acceptable carrier.

The term “unit dosage form” as used herein, refers to physically discrete units suitable as unitary dosages for human and/or animal subjects, each unit containing a predetermined quantity of the amino acid/derivative(s) calculated in an amount sufficient to produce the desired angiogenic and/or vasculogenic effect in association with a pharmaceutically acceptable diluent, carrier or vehicle. The specifications for the unit dosage forms of the present invention depend on the particular compound employed and the effect to be achieved, and the pharmacodynamics associated with each compound in the host.

The pharmaceutically acceptable excipients such as vehicles, adjuvants, carriers or diluents are readily available. Moreover pharmaceutically acceptable auxiliary substances, such as pH adjusting and buffering agents, tonicity adjusting agents, stabilizers, wetting agents & the like, are readily available.

In addition to the cationic amino acid and/or derivative(s), the pharmaceutical formulations according to the invention can comprise or be administered in parallel with agents that enhance angiogenesis by enhancing nitric oxide (NO) levels or prostacyclin levels.

Alternatively or in addition, the pharmaceutical compositions according to the invention can comprise additional angiogenesis inducing and/or vasculogenesis-inducing agents that act through other independent pathways (e.g., VEGF, FGF-a and FGF-b, etc.)

Particularly where the amino acid/derivative(s) are to be delivered for local applications, e.g., by intramuscular route, it may be desirable to provide the active ingredient(s) in a gel or matrix that can support angiogenesis, e.g., migration and proliferation of vascular cells into the matrix with endothelial tube formation. The gel or matrix can thus provide at least the initial substrate upon which new vessels form. For example, the gel or matrix can be extruded into an ischaemic or hypoxic region to form a path for new blood vessel formation so as to bypass an obstruction in the area.

### Induction of Angiogenesis In-Vivo

In order to accomplish stimulation of angiogenesis in vivo (e.g., as in the context of therapeutic angiogenesis), lysine and/or derivative(s) can be administered in any suitable form/manner, preferably with pharmaceutically acceptable carrier(s). One skilled in the art will readily appreciate that a variety of suitable methods of administering lysine and/or derivative(s), in the context of the present invention to a subject are available, and although, more than one route can be used to administer a particular compound a particular route can provide a more immediate, more effective and/or is associated with fewer side effects than another one or more routes. In general, lysine and/or derivative(s) can be administered according to the method of the invention by, for example, a parenteral, intravenous, intra-arterial, intrapericardial, intramuscular, intraperitoneal, transdermal, transcutaneous, subdermal, intradermal or intrapulmonary route.

Lysine and/or its derivatives can be administered before, during and/or after anticancer therapy involving, for example, administration of anticancer drug, radiation, hormonal, and/or gene therapy to which the cancer is susceptible. It is appreciated and understood that the angiogenic agent, such as lysine and/or its derivative(s), can be administered in combination, e.g., simultaneously, sequentially or separately, with one or more therapeutically active e.g., anti-tumor, compounds or one more therapeutically effective anticancer treatments.

Local administration can be accomplished by direct injection (e.g., intramuscular injection) at the desired treatment site, by introduction of the amino acid/derivative(s) formulations intravenously at a site near a desired treatment site (e.g., into a vessel or capillary that feeds a treatment site), by intra-arterial or intra-pericardial introduction, by introduction (e.g., by injection or other method of implantation) of lysine/derivative(s) formulation(s) in a biocompatible gel or capsule within or adjacent to a treatment site, by injection directly into muscle or other tissue in which increased blood flow and/or increased vascularity is desired, by rectal introduction of the formulation(s) (e.g., in the form of suppository to, for example, facilitate vascularization of a surgically created anastomosis after resection of a piece of bowel etc.).

In one particular application of interest, the lysine/derivative(s) formulation is employed in a bio-bypass method, wherein instead of performing a more invasive procedure, lysine/derivative(s) formulation(s) is administered to induce growth of new blood vessels around the blocked region. In this embodiment, the lysine/derivative(s) formulation can be administered in the area of and/or proximal to the treatment site to stimulate angiogenesis.

In some embodiments, it may be desirable to deliver the lysine/derivative(s) formulation directly to the wall of a vessel. One exemplary method of vessel-wall administration involves the use of a drug delivery catheter, particularly a drug delivery catheter comprising an inflation balloon that can facilitate delivery to a vessel wall. Thus, in one embodiment the method of the invention comprises delivery of lysine and/or derivative(s) to a vessel wall by inflating a balloon catheter wherein the balloon comprises one or more lysine/derivative(s) formulation covering a substantial portion of the balloon. The lysine/derivative(s) formulation(s) is held in place against the vessel wall promoting adsorption through the vessel wall. In one example the catheter is a perfusion balloon catheter, which allows perfusion of blood through the catheter while holding the lysine/derivative(s) against the vessel walls for longer adsorption times. Examples of catheters suitable for lysine/derivative(s) application include drug delivery catheters disclosed in U.S. Pat. Nos. 5,558,642 5,554,119 and 5,591,129.

In another embodiment, the lysine/derivative(s) formulation is delivered in the form of a biocompatible gel, which can be implanted (e.g., by injection into or adjacent a treatment site, by extrusion into or adjacent a tissue to be treated etc.) Gel formulations comprising lysine/derivative(s) can be designed to facilitate local release of the amino acid/derivative(s) and other active agents for a sustained period (e.g., over a period of hours, days, weeks, etc.). The gel can be injected into or near a treatment site, e.g., using a needle or other delivery device. In one embodiment, the gel is placed into or on an instrument which is inserted into the tissue and then slowly withdrawn to leave a track of gel, resulting in the stimulation of angiogenesis along the path made by the instrument. This latter method of delivery may be particularly desirable for the purpose of directing course of the bio-bypass.

In other embodiments, it may be desirable to deliver the lysine/derivative(s) formulation(s) topically, e.g., for localized delivery. Topical application can be accomplished by use of a biocompatible gel, which may be provided in the form of a patch, or by use of a cream, foam and the like. In general, topical administration is accomplished using a carrier such as a hydrophilic colloid or other material that provides a moist environment. An example of such an application would be as a sodium carboxymethyl cellulose based topical gel containing the lysine/derivative(s) and other ingredients together with preservatives and stabilizers.

In other embodiments, the lysine/derivative(s) formulation is delivered locally or systemically, preferably locally, using a transdermal patch. Several transdermal patches are well known in the art and such patches may be modified to provide for delivery of an amount of lysine/derivative(s), effective to stimulate angiogenesis according to the invention (see, e.g., U.S. Pat. Nos. 4,920,989 and 4,943,435).

In other methods of delivery, the lysine/derivative(s) can be administered using iontophoretic techniques. Methods and compositions for use in iontophoresis are well know in the art (see, e.g., U.S. Pat. Nos. 5,415,629 5,899,876 and 5,807,306).

The desirable extent of angiogenesis will depend on the particular condition or disease being treated, as well as the stability of the patient and possible side effects. In proper doses and with suitable administration, the present invention provides for a wide range of development of blood vessels, e.g., from little development to essentially full development.

Lysine/derivative(s) based formulations mediated stimulation of angiogenic response in tissues, where reperfusion is desired, is a phenomenon which is auto regulated in-vivo. The bi-amino cationic amino acid/derivative(s) exert its angiogenic response by bridging the growth factors (angiogenic factors) to their cell surface receptors. In one embodiment, the entire process of enhancement of angiogenesis according to the present invention is entirely dependent on the availability of the amino acid/derivative(s) in a relatively high concentration in the ischaemic or hypoxic zone/in its immediate vicinity, as well as on the presence/availability of naturally occurring angiogenic factor(s) in the zone of ischaemic or hypoxia or in the immediate vicinity.

### Dose

The dose of lysine/derivative(s) administered to a subject, particularly a human, in the context of the present invention should be sufficient to effect a therapeutic angiogenic response in the subject over a reasonable time frame. The dose will be determined by the condition of the subject, as well as the body weight of the subject as well as the level of angiogenic growth factor(s) in an ischaemic or hypoxic zone/area of tissue (e.g., hypoxic tumor matrix), number of available receptor sites (for these angiogenic factors) at a given moment of ischaemia or hypoxic and the level of proton concentration ([H+]) (degree of ischaemia or hypoxia) in the ischaemic or hypoxic zone. The cationic amino acid being non-toxic, non-mutagenic, extremely biocompatible, loading doses have also been reported without any side effects/adverse effects.

In determining the effective amount of lysine/derivative(s) in the stimulation of angiogenesis, the route of administration, the kinetics of the release system (e.g., pill, gel or other matrix) and the potency of the active ingredient(s) is considered so as to achieve the desired angiogenic effect(s) with minimal adverse side effect(s). The amino acid formulation(s) will typically be administered to the subject being treated for a time period ranging from a day to few weeks, consistent with the clinical conditions(s).

The following dosages assume that lysine is being administered or a lysine derivative with similar potency and efficiency as lysine. As will be readily apparent to the ordinarily skilled artisan, the dosage can adjusted for lysine derivative(s) according to their potency and/or efficacy relative to lysine. If given orally, the dose may be in the range of 10 mg to 400 mg given 1 to 20 times daily and can be up to a total daily dose of about 10 mg to 8 mg. If applied topically, to provide a local angiogenic effect, the dose would likely be in the range of about 0.001 mg to 10 mg per sq.cm surface area. If injected for the purpose of a local effect, the matrix is designed to release locally an amount of cationic amino acid equivalent (I-,d-, and oligo-lysine is/are equally effective, on their own, in inducing angiogenesis response) in the range of about 0.001 mg to 500 mg/unit area at the peak of activity. If injected for the purpose of a systemic effect, the matrix in which the lysine/derivative(s) is administered is designed to provide for a systemic delivery of a dose in the range of about 0.001 mg to 500 mg/dL of blood. If applied topically, for the purpose of a systemic effect, the patch or cream or gel (or any other suitable skin formulation) would be designed to provide for systemic delivery of a dose in the range of about 0.001 mg to 500 mg/dL of blood.

Regardless of the route of administration, the dose of lysine/derivative(s) can be administered over any appropriate time period, e.g., over the course of 1 to 24 hrs, over 1 to several days, etc. Furthermore, multiple doses can be administered over a selected time period. A suitable dose can be administered in suitable sub-doses per day, particularly in a prophylactic regimen. The precise treatment level will be dependent upon the response of the subject being treated. In the treatment of some individuals with lysine/derivative(s), it may be desirable to utilize a megadosing regimen. In such a treatment, a large dose of the lysine/derivative(s) is administered to an individual, and with time, the active ingredient(s) get eliminated through mainly two routes: a) rapid excretion through kidney, because of low mol. wt. and b) rapidly taken up by the cells for their metabolic needs. For the d-isomer, the molecule gets eliminated quickly unaltered because of its non-utilization in physiological system and low molecular weight.

### Condition Amenable to Treatment by Lysine/Derivative(s)-Mediated Induction of Angiogensis

The methods and lysine/derivative(s)-comprising composition of the invention can be use to treat a variety of conditions that would benefit from stimulation of angiogenesis, stimulation of vasculogenesis, increased blood flow, and/or increased vascularity.

Examples of conditions and diseases amenable to treatment according to the method of the invention include any condition associated with inadequate oxygen and/or blood supply. Specific examples of such conditions or diseases include but are not necessarily limited to tumors that are hypoxic or contain hypoxic tumor cells. Examples of such tumors include, but are not limited to, adenocarcinomas, glioblastomas (and other brain tumors), breast, cervical, colorectal, endometrial, gastric, liver, lung (small cell and non-small cell), lymphomas (including non-Hodgkin's, Burkitt's, diffused large cell, follicular and diffuse Hodgkin's), melanoma (metastatic), neuroblastoma, osteogenic sarcoma, ovarian, retinoblastoma, soft tissue sarcomas, head, neck, testicular and other tumors which respond to chemotherapy. Thus, the methods of the present invention can be used to treat cancer tumors, including experimental-induced cancer tumors, in any type of mammal including humans, commonly used laboratory animals such as rats, mice, rabbits and dogs, primates such as monkeys, and horses, cats and other animals.

The following examples are put forth so as to provide those of ordinary skill in the are with a complete disclosure and description of how to make and use the present invention, and are not intended to limit the scope of what the inventor regards as his invention nor are the intended to represent that the experiments below are all/or the only experiments performed. Efforts have been made to ensure accuracy with respect to numbers use (e.g. amounts, temperature, etc.) but some experimental errors and deviations should be accounted for.

### Example 1

### Oligo-Lysine and D-Lysine Induced Cell Growth and Angiogenesis In-Vitro and In-Vivo

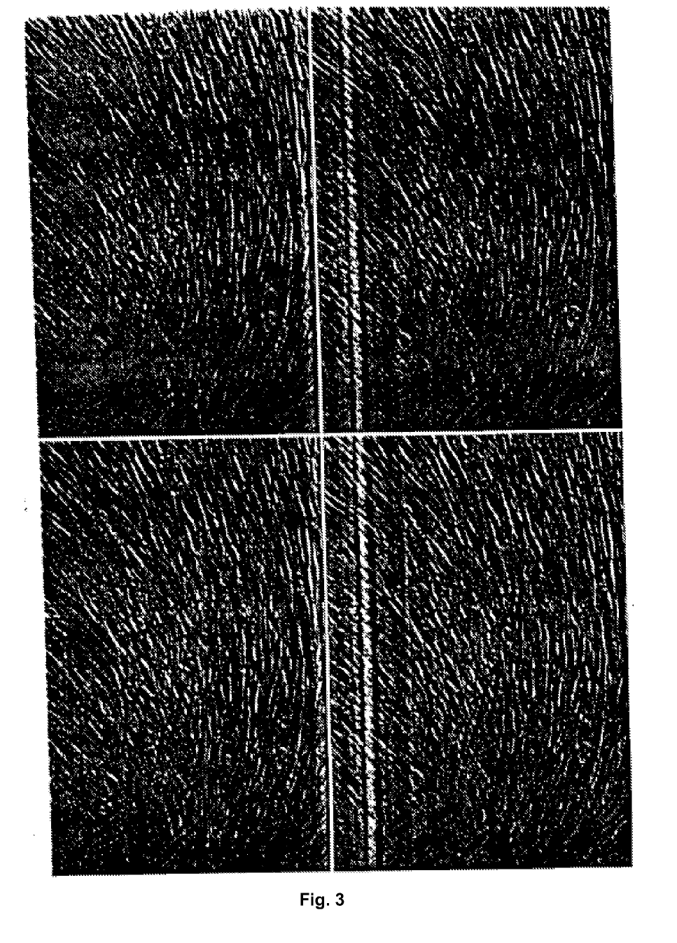

Lysine induced repair process depends on the ability of the amino acid/derivative(s) to induce rapid and controlled cellular expansion, both in-vitro and in-vivo. The phenomenon is probably based on the ability of the molecule(s) to act as the cell surface concentrator(s) of circulating growth factor(s). The rapid angiogenic property of the molecule(s) is also entirely dependent on the same phenomenon, where angiogenic factors are concentrated, locally, on the endothelial cell surface receptors, mediated by the molecule(s) working, possibly, as molecular bridges. Oligo-lysine and d-lysine are equally effective (as l-lysine) in inducing rapid cellular growth in-vitro (FIG. 3) and angiogenesis in-vivo.

### Example 2

### Lysine Facilitated Radiation Therapy

The following example is provide to illustrate treatment of cancer with lysine combination therapy. A Pharmaceutical lysine formulation is administered to cancer patients with cancer types (e.g. cervical carcinoma and head and neck tumors) that contain hypoxic regions.

The pharmaceutical lysine formulation is administered orally or parenterally. The formulation can contain L- or D-Lysine or both in native or activated form, with or without oligo-lysine of up to mol.wt. of about 5500. Further, the formulation can included suitable additive(s), adjuvant(s) and/or stabilizers, as appropriate. If the formulation contains more that on species of lysine, the species can be present in equal or unequal amounts.

In a treatment regimen, the pharmaceutical lysine formulation is administered over a period of first 6-7 days. In an alternative treatment regimen, the pharmaceutical lysine formulation is administered over a period of first 48-96 hours.

The dosage schedule is typically in the range of 4-6 g per day, but a higher dosage (up to a maximum of 15 g per day) may administered depending on the desired degree of angiogenic response or the angiogenic response obtained. The formulation can be given in one dose or divided doses.

In the first embodiments, pharmaceutical lysine formulation is co-administered with one or more suitable sensitizing agents to sensitize hypoxic tumor cells to therapeutic radiation therapy. The enhance vascular network induced by the angiogenic response of the lysine facilitated treatment increases the delivery or bioavailability of the sensitizing agent(s) to the hypoxic layers of tumor cells. Hence, the cell kill per cycle of radiation therapy is enhanced, whereby the number of viable cancer cells in the patients is reduced or even eliminated.

In one embodiment, the administration of the pharmaceutical lysine formulation results in a reduction in the dose of sensitizing agent and/or therapeutic radiation and/or in the number of radiotherapy cycles(s) that would otherwise be required without lysine—induced angiogenesis within the hypoxic layers of the tumor matrix.

In another embodiment, pharmaceutical lysine formulation is co-administered with radiation therapy and chemotherapy.

### Example 3

### Lysine Facilitated Cancer Therapy

The pharmaceutical lysine formulation in the same manner as described in Example 2 is co administered with one or more of the following suitable anticancer compounds: chemotherapeutic agent, hormone therapy agent, gene therapy agent monoclonal antibody agent and polyclonal antibody agent. The enhanced vascular network induced by the angiogenic response of the lysine facilitated treatment increases the delivery of bioavailability of the anticancer compound(s) to the hypoxic layers of tumor cells. Hence, the cell kill per cycle of cancer therapy is enhanced and the number of viable cancer cells in the patients is reduced or even eliminated.

### Example 4

### Lysine Facilitated Cancer Therapy

A 30 years male patient (PD) presented with an ulceroproliferative growth in the left cheek with ipsilateral neck node mass (T₃N₂M₀) in April, 2007. He underwent Radical surgery in the form of total excision of primary tumors and bilateral Supra-omohyoid Neck dissection. Adjuvant External Beam Radiotherapy (EBRT) was then given 66 Gy/33 Fr 6½ weeks and the patient went into complete remission and was disease free for >9 months.

Subsequently, the patient had multiple nodal recurrences presenting with palpable masses.

Left submandibular—5 to 6 cm (fixed)

### Left Supraclavicular—2 cm

Left Post Cervical—2 cm and also

### Right Submandibular—2 cm

Recurrences were confirmed with biopsy and HP examination.
Subsequently, the patient was given 2 cycles of Palliative Chemotherapy, following Injection LMH (Lysine Monohydrochloride) for 4 days (4 g/day I.V. in divided doses), with Cisplatin and 5 FU.

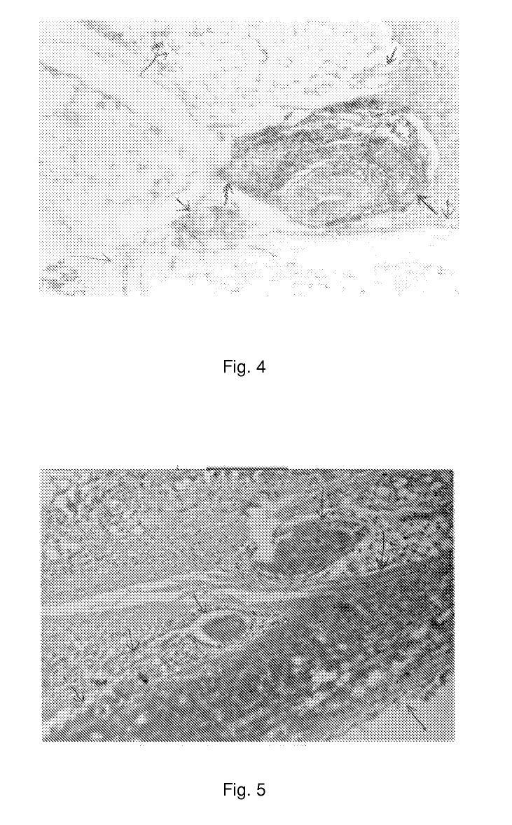

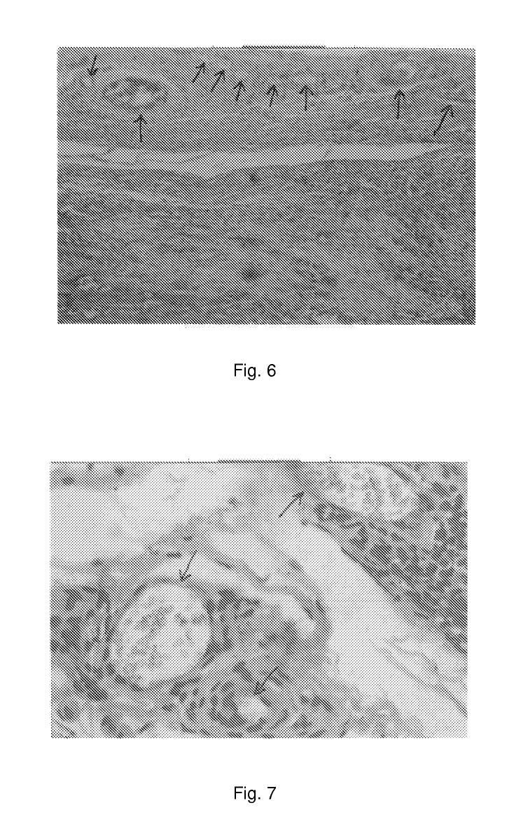

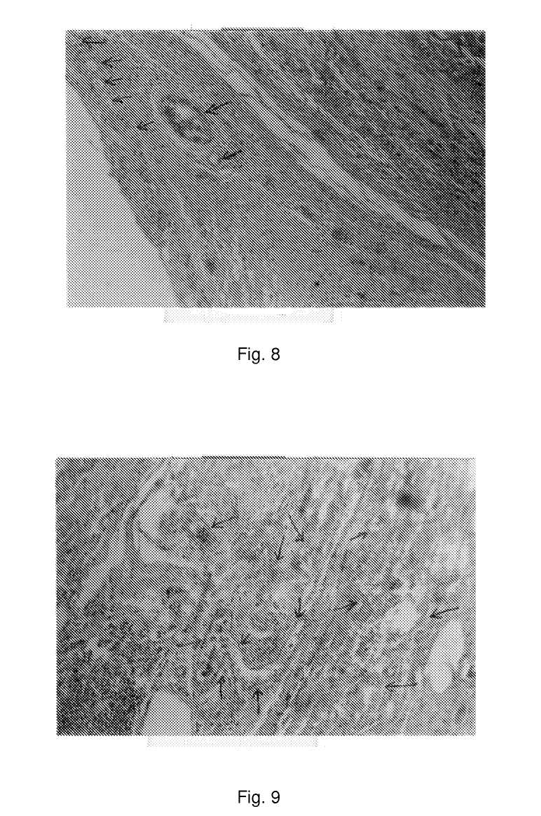

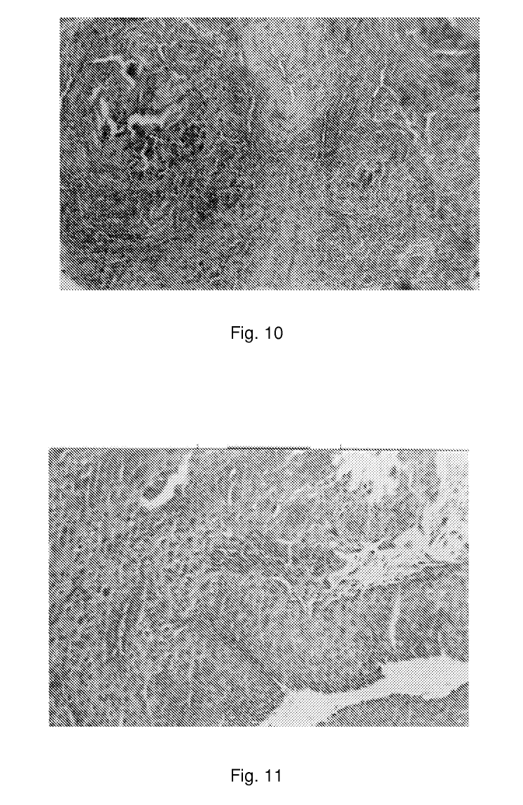

HPE was done 96 hrs after Injection LMH to confirm the neo-angiogenesis (FIGS. 4 & FIGS. 5 & FIGS. 6-9)(Also compare FIGS. 4 to 9 with FIGS. 10-11 for quantitative comparison of angiogenic response in LMH treated squamous cell carcinomatous tumor vs. control tumours).

The recurrence lymph node masses have disappeared achieving a 2^{nd} complete recovery.

Contemplating further 2-3 cycles of chemotherapy for better palliation and longer disease free period.

Non-limiting examples of anticancer agents suitable for use in the lysine-facilitated cancer treatment of the present invention include inter alia paclitaxel, carboplatin, cisplatin, and 5-FU, Amifostine, Bleomycin sulphate, Capecitabine, Carboplatin, Cisplatin, Cyclophosphamide, Docetaxel, Doxorubicin, Etoposide, Erythropoietin, Filgastrim, 5-Flurouracil, Gemcitabine, Hydroxyurea, Infosfamide with Mesna, Letrozole, Lencovorin Calcium, Methotrexate, Oxaliplatin, paclitaxel, pamidronate, Tamoxifen citrate, Temozolamide, Topofelan, Thalidomide, Vinorelbine, Vincristine, Vinblastine, Zoledronic acid, Procarbazine, Actinomycin-D, DTIC, Liposomal Doxorubicin, Procarbazine, Chlorambucil, Melphalan, Methylprednisolone, Goserelin acetate, Lenprolide acetate, Anastrozole, Her-2-neu MAb, Anti EGFR MAb, Tyrosine Kinase MAb, Anti VEGF MAb. Some are preferentially used to treat certain cancer types over others. Frequently, more than one agent can be used in combination chemotherapy cycles. In many cases, the anticancer agents, such as those mentioned above, are used in combination with radiotherapy. In those situation, the anticancer agents also act as radio-sensitizer agents or drugs. Examples of drugs that sensitize tumor cell to anticancer treatment (e.g., radiotherapy) include misonidazole and mitomycin.

While this invention has been described in detail and with reference to specific embodiments thereof, it will be apparent to one skilled in the art that various changes and modifications can be made therein without deviating or departing from the scope and spirit of the invention. Thus the disclosure contained herein includes within its ambit obvious equivalents and substitutes as well.

Having described the invention in details with particular reference to the illustrative examples given above, it will now more specifically defined by means of claims appended hereafter.

What is claimed is:

1. A method of treating hypoxic tumor cells in a tumor in a subject, comprising the steps of:
administering an angiogenic inducing agent to the subject in an amount effective to promote a desired degree of vascularization within the tumor or a selected portion thereof containing the hypoxic tumor cells; and
administering a tumor sensitizing agent to the subject in an amount effective to produce an increase in sensitivity of the hypoxic tumors cells to a tumorcidal effect of a cancer treatment to which the hypoxic tumor cells are susceptible,
wherein vascularization of the hypoxic tumor cells increases bioavailability of the tumor sensitizing agent to the hypoxic tumor cells.

2. The method of claim 1, wherein the angiogenic agent is lysine or a pharmaceutically acceptable salt thereof.

3. The method of claim 2, further comprising at least one of the pharmaceutically acceptable carrier, adjuvant, diluent, additive stabilizer and exipient.

4. The method of claim 2, wherein lysine is L-Lysine, D-Lysine, an activated L-lysine, an activated D-lysine, an oligo-lysine or a mixture of at least two of the foregoing.

5. The method of claim 4, wherein the oligo-lysine has a molecular weight of up to about 5500.

6. The method of claim 2, wherein lysine is administered to the subject over a period of 6-7 days.

7. The method of claim 2, wherein lysine is administered to the subject over a period of 48-96 hours.

8. The method of claim 2, wherein up to 15 g of lysine is administered to the subject per day.

9. The method of claim 2, wherein up to 4-6 g of lysine is administered to the subject per day.

10. The method of claim 2, wherein lysine is administered to the subject orally, parenterally, intravenously, subcutaneously or intramusculary.

11. The method of claim 2, wherein lysine is administered locally to into an area of the hypoxic tumor cells.

12. The method of claim 1, wherein at least two angiogenic inducing agents of substantially equal amounts are administered to the subject.

13. The method of claim 1, wherein the tumor sensitizing agent is a radiosensitizing agent, wherein the cancer treatment is radiotherapy, wherein the radiosensitizing agent produces an increase in sensitivity of hypoxic tumor cells to ionizing radiation administered during radiotherapy, and wherein vascularization of the hypoxic tumor cells reduces an amount of the ionizing radiation required to achieve tumorcidal effect.

14. The method of claim 13, wherein the radiation is administered in amount effective to reduce a volume of the hypoxic tumor cells.

15. The method of claim 13, wherein the cancer treatment further comprising chemotherapy.

16. The method of claim 1, wherein the angiogenic inducing agent is administered before, after or concurrently with the tumor sensitizing agent.

17. The method of claim 1, wherein the angiogenic inducing agent is administered before, after or concurrently with radiation and/or a chemotherapy agent.

18. The method of claim 1, wherein the tumor is prostate tumor, breast tumor, epithelial tumor, lung tumor, colon tumor, leukemia, melanoma, ovarian tumor, adenocarcinoma, myeloma, sarcoma, or rectum cancer.

19. The method of claim 1, wherein the subject is a human being.

20. The method of claim 1, wherein the cancer treatment comprises administering a therapeutically effective amount of a chemotherapeutic agent, and wherein the effective amount of the chemotherapeutic agent is an amount less than that required to achieve tumorcidal effect without vascularization.

21. A method of treating hypoxic tumor cells in a tumor in a subject, comprising the steps of:
administering an angiogenic inducing agent to the subject in an amount effective to promote a desired degree of vascularization with the tumor or a selected portion thereof containing the hypoxic tumor cells; and
administering a therapeutically effective amount of at least one of a chemotherapeutic agent, a gene therapy agent, a hormone therapy agent, a monoclonal antibody and a polyclonal antibody to which the tumor is susceptible to the subject,
wherein vascularization of the hypoxic tumor cells increases bioavailability of the chemotherapeutic agent, gene therapy agent, hormone therapy agent, monoclonal antibody and/or polyclonal antibody to the hypoxic tumor cells.

22. The method of claim 21, wherein the angiogenic agent is lysine or a pharmaceutically acceptable salt thereof.

23. The method of claim 22, further comprising at least one of a pharmaceutically acceptable carrier, adjuvant, diluent, additive stabilizer and exipient.

24. The method of claim 22, wherein lysine is L-lysine, D-lysine, an activated L-lysine, an activated D-lysine, an oligolysine or a mixture of at least two of the foregoing.

25. The method of claim 24, wherein the oligolysine has a molecular weight of up to about 5500.

26. The method of claim 22, wherein lysine is administered to the subject over a period of 6-7 days.

27. The method of claim 22, wherein lysine is administered to the object over a period of 48-96 hours.

28. The method of claim 22, wherein up to 15 g of lysine is administered to the subject per day.

29. The method of claim 22, wherein up to 4-6 g of lysine is administered to the subject per day.

30. The method of claim 22, wherein lysine is administered to the subject orally, parenterally, intravenously, subcutaneously or intramuscularly.

31. The method of claim 22, where lysine is administered locally to into an area of the hypoxic tumor cells.

32. The method of claim 21, wherein at least two angiogenic inducing agents of substantially equal amounts are administered to the subject.

33. The method of claim 21, further comprising administering a therapeutically effective amount of ionizing radiation, wherein vascularization of the hypoxic cells reduces an amount of radiation required to achieve an effective tumorcidal effect.

34. The method of claim 21, wherein the angiogenic inducing agent is administering before, after or concurrently with the tumor sensitizing agent.

35. The method of claim 21, wherein the angiogenic inducing agent is administered before, after or concurrently the chemotherapeutic agent.

36. The method of claim 21, wherein the tumor is prostate tumor, breast tumor, epithelial tumor, lungs tumor, colon tumor, leukemia, melanoma, ovarian tumor, adenocarcinoma, myeloma, sarcoma, or rectum cancer.

37. The method of claim 21, wherein the subject is a human being.

38. The method of claim 21, wherein the effective amount of the chemotherapeutic agent, gene therapy agent, hormone therapy agent, monoclonal antibody and/or polyclonal antibody is an amount less than that required to achieve tumorcidal effect without vascularization.

39. A therapeutic composition for treating cancer comprising lysine, or one or more derivatives thereof as shown in FIGS. 1A, 1B, 1C and 1D of the drawings in dosage amount varying between 1 and 15 g per day, along with at least one sensitizing agent(s)/chemotherapeutic agent(s) in an amount varying between 150 mg and 1000 mg.

40. A composition of claim 39, wherein a formulation containing at least lysine of any of the form is co-administered with chemotherapy and/or radiotherapy

41. A composition of claim 39, wherein a formulation containing at least one form of lysine is co-administered with one or more suitable sensitizing agent(s) in its/their therapeutic doses.

42. A Composition of claim 39, wherein there is used a mixture of at least two different forms of Lysine selected from the group of L- or D-Lysine, and activated D-Lysine, an activated L-Lysine, and oligo-Lysine or pharmaceutically acceptable salt thereof.

43. A Composition of claim 39, wherein the composition comprises a lysine and or derivative(s) thereof selected from FIG. 1A, 1B, 1C or 1D of the drawings, with Xylitol in an amount varying between 0.5% (wt/vol) and 50% (wt/vol.).

44. A Composition of claim 43, wherein Xylitol is present in an amount varying between 0.5% (wt/vol) and 10.0% (wt/vol.)

45. A Composition of claim 43, wherein lysine or derivatives thereof selected from FIGS. 1A, 1B, 1C and 1D of the drawings is used in aqueous medium varying between 1 gm and 50 gm per diem in divided doses.

46. A Composition of claim 45, wherein lysine is preferably used in an amount varying between 4 gm and 20 gm per diem in divided doses.
